(19) United States
(12) Patent Application Publication
(10) Pub. No.: US 2024/0023812 A1
(43) Pub. Date: Jan. 25, 2024

(54) PHOTOGRAPHING SYSTEM THAT ENABLES EFFICIENT MEDICAL EXAMINATION, PHOTOGRAPHING CONTROL METHOD, AND STORAGE MEDIUM

(71) Applicant: CANON KABUSHIKI KAISHA, Tokyo (JP)

(72) Inventor: Sho ICHIKAWA, Kanagawa (JP)

(21) Appl. No.: 18/351,596

(22) Filed: Jul. 13, 2023

(30) Foreign Application Priority Data
Jul. 22, 2022 (JP) 2022-117336

(51) Int. Cl. A61B 5/00; H04N 23/69; H04N 23/661; G16H 40/67; G16H 50/20

(52) U.S. Cl. CPC A61B 5/0077; H04N 23/69; H04N 23/661; G16H 40/67; G16H 50/20

(57) ABSTRACT

A photographing system that enables an attending doctor to perform efficient medical examination. The photographing system supports photographing of an affected area using an image capturing apparatus. Photographing control information to be used by the image capturing apparatus to photograph the affected area is acquired by inputting disease information transmitted from the image capturing apparatus to a learned model, and is transmitted to the image capturing apparatus. An affected area image is acquired which is generated by the image capturing apparatus photographing the affected area. In a case where the acquired affected area image is an image generated by manual photographing for performing photographing using other photographing control information adjusted from the photographing control information, relearning of the learned model is performed based on information on the acquired affected area image.

### BACKGROUND OF THE INVENTION

### Field of the Invention

The present invention relates to a photographing system that enables efficient medical examination, a photographing control method, and a storage medium.

### Description of the Related Art

In a medical practice, to observe a process of diagnosis and medical treatment, an affected area image produced by photographing an affected area is recorded. As a method of easily acquiring an affected area image that facilitates diagnosis, for example, there has been proposed a technique in which an image capturing apparatus identifies a region of an affected area in a live view image using a learned model and automatically photographs the affected area at a timing when the size of a region of the affected area becomes equal to a predetermined size or larger (see e.g. Japanese Laid-Open Patent Publication (Kokai) No. 2020-156082).

However, the above-described technique disclosed in Japanese Laid-Open Patent Publication (Kokai) No. 2020-156082 has a problem that, depending on a disease type, it is sometimes impossible to acquire an affected area image that facilitates diagnosis, so that an attending doctor cannot perform efficient medical examination. For example, in a disease having symptoms including a rash, such as hives, features of the affected area are sometimes fine and widespread, which makes a necessary diagnostic image region different depending on an attending doctor. Therefore, the attending doctor checks an affected area image obtained by automatically photographing the affected area using the learned model, and when the doctor judges that the affected area image is unsuitable for diagnosis, the doctor manually photographs the affected area again. Thus, conventionally, depending on a disease type, it is necessary to manually photograph an affected area again each time, which prevents the attending doctor from performing efficient medical examination.

### SUMMARY OF THE INVENTION

The present invention provides a photographing system that enables an attending doctor to perform efficient medical examination, a photographing control method, and a storage medium.

In a first aspect of the present invention, there is provided a photographing system that supports photographing of an affected area using an image capturing apparatus, including at least one processor, and a memory coupled to the at least one processor, the memory having instructions that, when executed by the processor, perform the operations as: a first acquisition unit configured to acquire photographing control information to be used by the image capturing apparatus to photograph the affected area, by inputting disease information transmitted from the image capturing apparatus to a learned model, a transmission unit configured to transmit the photographing control information to the image capturing apparatus, a second acquisition unit configured to acquire an affected area image generated by the image capturing apparatus photographing the affected area, and a relearning unit configured to perform, in a case where the acquired affected area image is an image generated by manual photographing for performing photographing using other photographing control information adjusted from the photographing control information, relearning of the learned model based on information on the acquired affected area image.

In a second aspect of the present invention, there is provided a photographing control method for supporting photographing of an affected area using an image capturing apparatus, including acquiring photographing control information to be used by the image capturing apparatus to photograph the affected area, by inputting disease information transmitted from the image capturing apparatus to a learned model, transmitting the photographing control information to the image capturing apparatus, acquiring an affected area image generated by the image capturing apparatus photographing the affected area, and performing, in a case where the acquired affected area image is an image generated by manual photographing for performing photographing using other photographing control information adjusted from the photographing control information, relearning of the learned model based on information on the acquired affected area image.

According to the present invention, the attending doctor is enabled to perform efficient medical examination.

Further features of the present invention will become apparent from the following description of exemplary embodiments (with reference to the attached drawings).

### BRIEF DESCRIPTION OF THE DRAWINGS

FIG. 1 is a diagram showing an example of a photographing system according to the present embodiment.

FIG. 2 is a block diagram showing a hardware configuration of the photographing system shown in FIG. 1.

FIG. 3 is a block diagram showing a software configuration of the photographing system shown in FIG. 1.

FIG. 4 is a flowchart of a learned model generation process performed in the photographing system shown in FIG. 1.

FIG. 5 is a diagram useful in explaining learning processing in a step in FIG. 4.

FIG. 6 is a diagram showing a learned model generated in the photographing system shown in FIG. 1.

FIG. 7A is a view showing an overhead image of an affected area, which is obtained by photographing the whole affected area of a patient suffering from hives.

FIG. 7B is a view showing an enlarged image of the affected area, which is obtained by photographing part of the affected area of the patient suffering from hives.

FIG. 8A is a flowchart of a photographing control process performed in the photographing system shown in FIG. 1.

FIG. 8B is a continuation of FIG. 8A.

FIG. 9A is a view showing an example of an enlarged image captured from part of an affected area of a patient suffering from hives.

FIG. 9B is a view showing another example of the enlarged image captured from part of the affected area of the patient suffering from hives.

FIG. 10 is a diagram useful in explaining relearning processing in a step in FIG. 8B.

### DESCRIPTION OF THE EMBODIMENTS

The present invention will now be described in detail below with reference to the accompanying drawings showing embodiments thereof.

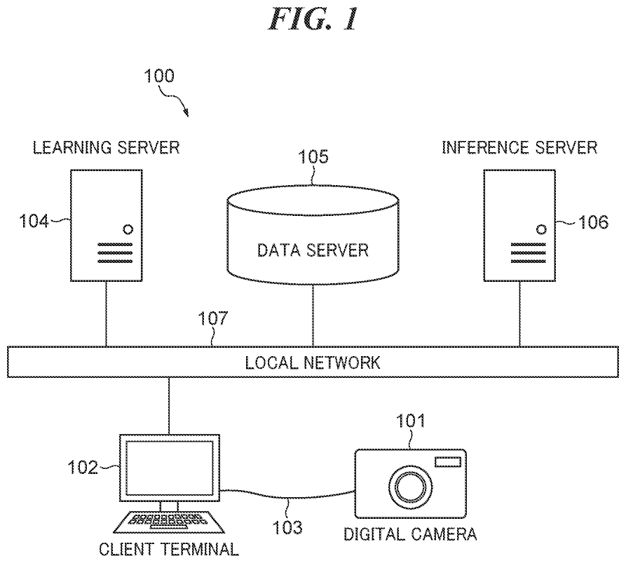

FIG. 1 is a diagram showing an example of a photographing system 100 according to the present embodiment. A digital camera 101 is an electronic apparatus used by a user. Hereafter, the description is given assuming that the electronic apparatus included in the photographing system 100 according to the present embodiment is the digital camera 101 (image capturing apparatus), but the present embodiment can be applied to a desired electronic apparatus. For example, the electronic apparatus may be any of apparatuses having a photographing function, such as a smartphone, a tablet terminal, and a personal computer (PC). The digital camera 101 is communicably connected to a client terminal 102 via communication means 103. The communication via the communication means 103 may be wired communication or wireless communication.

A learning server 104 is a learning apparatus that is capable of causing a learning model to be machine-learned. Hereafter, the description is given assuming that the learning server 104 performs deep learning as the machine learning. However, the machine learning performed by the learning server 104 is not limited to deep learning. For example, the learning server 104 may perform machine learning using a desired machine learning algorithm, such as a decision tree or a support vector machine.

A data server 105 stores a variety of types of data. For example, the data server 105 stores data for learning, which is used when the learning server 104 performs machine learning. An inference server 106 performs inference processing using a learned model generated by the learning server 104. The client terminal 102, the learning server 104, the data server 105, and the inference server 106 are mutually communicably connected via a local network 107.

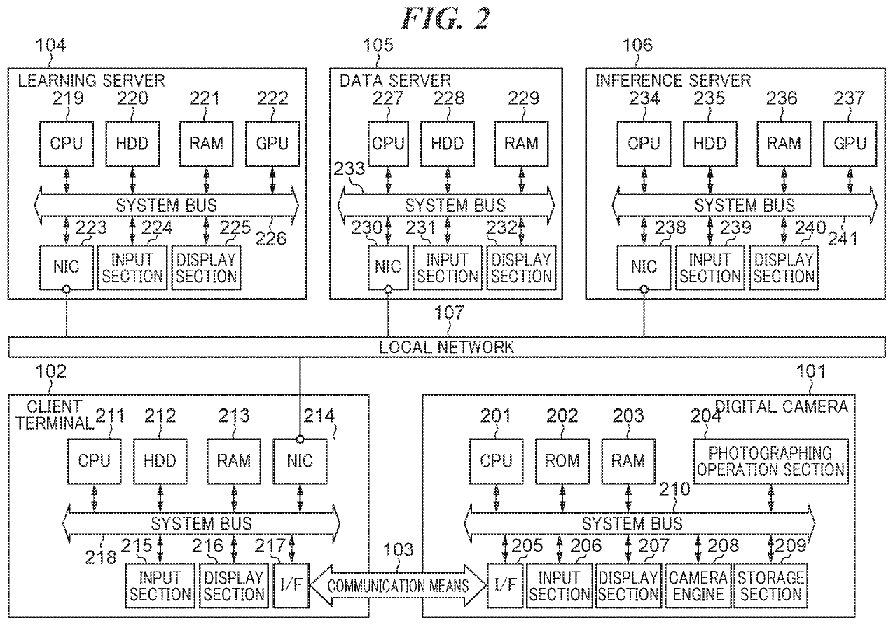

FIG. 2 is a block diagram showing a hardware configuration of the photographing system shown in FIG. 1. First, a hardware configuration of the digital camera 101 will be described. A CPU 201 controls the overall operation of the digital camera 101. Further, the CPU 201 corresponds to a control unit configured to control power supply. A ROM 202 stores programs and data for the operation of the CPU 201. A RAM 203 is a memory for loading a program read by the CPU 201 from the ROM 202 and temporarily storing data used for the operation. A photographing operation section 204 performs photographing according to a photographing instruction received from a user. An interface 205 is for exchanging data between the digital camera 101 and the client terminal 102 via the communication means 103.

An input section 206 is comprised of an image sensor, a motion sensor, and the like. The image sensor is used by the digital camera 101 to perform photographing. The motion sensor detects a motion which requires camera shake correction. Further, the input section 206 has a function of receiving an instruction from a user. For example, the input section 206 receives e.g. an instruction input using a switch for designating an operation mode of the digital camera 101.

A display section 207 can display an image which is being photographed or has been photographed by the image sensor of the input section 206. Further, the display section 207 can also display an operation state of the camera. A camera engine 208 processes an image captured by the image sensor of the input section 206. Further, the camera engine 208 performs image processing for displaying an image stored in a storage section 209 on the display section 207. The storage section 209 stores still images and moving images photographed by the digital camera 101. A system bus 210 connects the blocks forming the digital camera 101.

Next, a hardware configuration of the client terminal 102 will be described. A CPU 211 controls the overall operation of the client terminal 102. An HDD 212 stores programs and data for the operation of the CPU 211. A RAM 213 is a memory for temporarily storing a program read by the CPU 211 from the HDD 212 and data used for the operation of the CPU 211. An NIC 214 is an interface card for communicating with the data server 105 and the inference server 106 via the local network 107. An input section 215 is comprised of a keyboard and a mouse for operating the client terminal 102. A display section 216 displays information input to the client terminal 102, and the like. The display section 216 is e.g. a display. An interface 217 is for exchanging data between the client terminal 102 and the digital camera 101 via the communication means 103. A system bus 218 connects the blocks forming the client terminal 102.

Next, a hardware configuration of the learning server 104 will be described. A CPU 219 controls the overall operation of the learning server 104. An HDD 220 stores programs and data for the operation of the CPU 219. A RAM 221 is a memory for temporarily loading a program read by the CPU 219 from the HDD 220 and data used for the operation of the CPU 219.

A GPU 222 is an integrated circuit specialized for arithmetic data processing so as to make it possible to process a lot of data items in parallel by performing arithmetic operation for image processing, matrix operation, and the like, at high speed. Accordingly, the GPU 222 is suitably used for a case where learning processing is executed a plurality of times on a learning model, as executed in deep learning. Note that in the present embodiment, the learning processing performed by the learning server 104 is performed by cooperation of the CPU 219 and the GPU 222. More specifically, the CPU 219 and the GPU 222 perform arithmetic operations of a learning program including a learning model in cooperation, thereby performing learning processing. Note that one of the CPU 219 and the GPU 222 may perform the learning processing.

An NIC 223 is an interface card for communicating with the data server 105 and the inference server 106 via the local network 107. An input section 224 is comprised of a keyboard and a mouse for operating the learning server 104. A display section 225 displays information input to the learning server 104, and the like. The display section 225 is e.g. a display. A system bus 226 connects the blocks forming the learning server 104.

Next, a hardware configuration of the data server 105 will be described. A CPU 227 controls the overall operation of the data server 105. An HDD 228 stores programs and data for the operation of the CPU 227. A RAM 229 is a memory for temporarily loading a program read by the CPU 227 from the HDD 228 and data used for the operation of the CPU 227. An NIC 230 is an interface card for communicating with the client terminal 102 and the learning server 104 via the local network 107. An input section 231 is comprise of a keyboard and a mouse for operating the data server 105. A display section 232 displays information input to the data server 105, and the like. The display section 232 is e.g. a display. A system bus 233 connects the blocks forming the data server 105.

Next, a hardware configuration of the inference server 106 will be described. A CPU 234 controls the overall operation of the inference server 106. An HDD 235 stores programs and data for the operation of the CPU 234. A RAM 236 is a memory for temporarily loading a program read by the CPU 234 from the HDD 235 and data used for the operation of the CPU 234.

Similar to the GPU 222, a GPU 237 is an integrated circuit capable of processing a lot of data items in parallel by performing arithmetic operation for image processing, matrix operation, and the like, at high speed. Accordingly, the GPU 237 is suitably used for a case where inference processing is performed using a learned model obtained by deep learning. In the present embodiment, the inference processing performed by the inference server 106 is performed by cooperation of the CPU 234 and the GPU 237. More specifically, the CPU 234 and the GPU 237 cooperate to perform arithmetic operations to thereby perform inference processing using a learned model. Note that the configuration may be such that one of the CPU 234 and the GPU 237 performs inference processing using the learned model. An NIC 238 is an interface card for communicating with the client terminal 102 and the learning server 104 via the local network 107. An input section 239 is comprised of a keyboard and a mouse for operating the inference server 106. A display section 240 displays information input to the inference server 106, and the like. The display section 240 is e.g. a display. A system bus 241 connects the blocks forming the inference server 106.

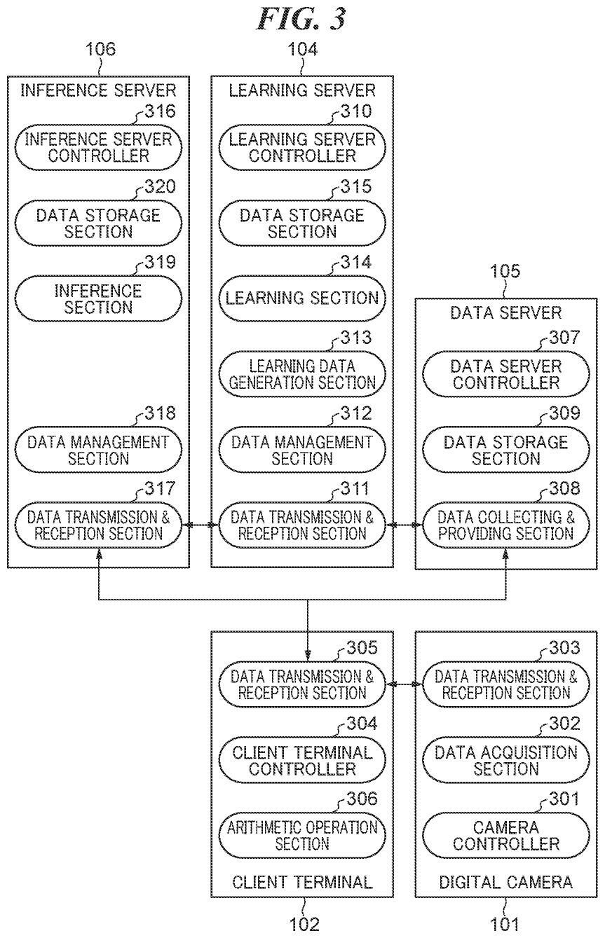

FIG. 3 is a block diagram showing a software configuration of the photographing system shown in FIG. 1. First, a software configuration of the digital camera 101 will be described. A camera controller 301 controls the overall operation of the digital camera 101. The camera controller 301 is realized by the CPU 201 executing a program loaded in the RAM 203. The camera controller 301 causes the camera engine 208 to process an input from the image sensor according to a user operation received by the photographing operation section 204 or the input section 206. Further, the camera controller 301 also performs control to display an image stored in the storage section 209 on the display section 207.

A data acquisition section 302 acquires an affected area image used for learning processing performed by the learning server 104. The affected area image is an image obtained through photographing of an affected area by the image sensor of the input section 206. Further, the data acquisition section 302 acquires disease information, described hereinafter, used for inference processing performed by the inference server 106. A data transmission and reception section 303 transmits the affected area image acquired by the data acquisition section 302 to the client terminal 102. Further, the data transmission and reception section 303 transmits the disease information acquired by the data acquisition section 302 to the client terminal 102. Further, the data transmission and reception section 303 receives photographing control information, described hereinafter, which is output by inference processing performed by the inference server 106 from the client terminal 102 via the interface 205.

Next, a software configuration of the client terminal 102 will be described. A client terminal controller 304 controls the overall operation of the client terminal 102. For example, it is assumed that the user inputs to the input section 215 an instruction for requesting transmission of data for learning, while viewing the display section 216. In this case, the client terminal controller 304 acquires the data for learning from the digital camera 101 and instructs transmission of the acquired data for learning to the data server 105 based on the instruction input to the input section 215. Further, let it be assumed that the user inputs to the input section 215 an instruction for requesting transmission of photographing control information output by inference processing performed by the inference server 106 while viewing the display section 216. In this case, the client terminal controller 304 receives the photographing control information from the inference server 106 and instructs transmission of the received photographing control information to the digital camera 101, based on the instruction input to the input section 215. The client terminal controller 304 is realized by the CPU 211 executing a program loaded in the RAM 213.

A data transmission and reception section 305 receives the data for learning, which is transmitted by the digital camera 101, via the interface 217 and transmits the received data for learning to the data server 105 via the NIC 214. Further, the data transmission and reception section 305 transmits the disease information transmitted by the digital camera 101 to the inference server 106. Further, the data transmission and reception section 305 receives photographing control information output by inference processing performed by the inference server 106 via the NC 214 and transmits the received photographing control information to the digital camera 101 via the interface 217. An arithmetic operation section 306 calculates an area of an affected area region and a difference in a photographed affected area region size, referred to hereinafter.

Next, a software configuration of the data server 105 will be described. A data server controller 307 controls the overall operation of the data server 105. For example, the data server controller 307 performs control to cause data for learning, which is received from the client terminal 102, to be stored in the HDD 228. Further, the data server controller 307 performs control to transmit the data for learning to the learning server 104, based on a request for transmitting the data for learning, which is received from the learning server 104. The data server controller 307 is realized by the CPU 227 executing a program loaded in the RAM 229. A data collecting and providing section 308 collects data for learning from the client terminal 102. Further, the data collecting and providing section 308 provides data for learning to the learning server 104 via the NIC 230. A data storage section 309 stores the data for learning, which is collected from the client terminal 102. Further, when providing the data for learning to the learning server 104, the data storage section 309 reads out the data for learning and passes the read data for learning to the NIC 230. The data storage section 309 is implemented by the HDD 228 or the like.

Next, a software configuration of the learning server 104 will be described. A learning server controller 310 controls the overall operation of the learning server 104. For example, let it be assumed that the user inputs to the input section 224 a learning processing instruction while viewing the display section 225. In this case, the learning server controller 310 performs control to acquire data for learning from the data server 105 based on the instruction input to the input section 224. Then, the learning server controller 310 causes a learning section 314 to perform machine learning. The learning server controller 310 performs control to transmit a learned model generated by the machine learning performed by the learning section 314 to the client terminal 102. The learning server controller 310 is realized by the CPU 219 executing a program loaded in the RAM 221.

A data transmission and reception section 311 receives the data for learning, which is transmitted from the data server 105, via the NIC 223. Further, the data transmission and reception section 311 transmits the learned model generated by the machine learning performed by the learning section 314 to the inference server 106 via the NIC 223.

A data management section 312 determines whether or not to use the data for learning, which is received by the data transmission and reception section 311. Further, the data management section 312 determines whether or not to transmit the learned model from the data transmission and reception section 311. Note that in the present embodiment, not all data items of the data for learning are used as input data to be input to the learned model, but some of the data items may be used as data for verification. As a method of dividing the data for learning into the input data to be input to the learned model and the data for verification, for example, a hold-out method can be applied. A learning data generation section 313 performs processing for dividing the data for learning into the input data to be input to the learned model and the data for verification, and the like. The data generated by this processing is stored in the RAM 221 or the HDD 220.

The learning section 314 performs machine learning of a learning model using the data for learning, which is stored in the RAM 221 or the HDD 220. The function of the learning section 314 can be realized by the CPU 219 or the GPU 222. When the machine learning of the learning model is completed, a learned model is obtained. A data storage section 315 stores the learned model obtained by the machine learning. The data storage section 315 is implemented by the HDD 220 or the like.

Next, a software configuration of the inference server 106 will be described. An inference server controller 316 controls the overall operation of the inference server 106. For example, in a case where a data transmission and reception section 317 has received disease information, described hereinafter, from the client terminal 102, the inference server controller 316 causes an inference section 319 to execute inference processing using a learned model. The inference server controller 316 is realized by the CPU 234 that executes a program loaded in the RAM 236. The data transmission and reception section 317 receives a learned model transmitted from the learning server 104 via the NIC 238. Further, the data transmission and reception section 317 receives disease information transmitted from the client termina 102. Further, the data transmission and reception section 317 transmits photographing control information output by inference processing, to the client terminal 102 via the NIC 238. A data management section 318 determines whether or not to use the learned model and the disease information, which are received by the data transmission and reception section 317, for inference processing. Further, the data management section 318 determines whether or not to transmit the photographing control information output by the inference processing. The inference section 319 performs inference processing by inputting the acquired disease information to the learned model. The inference section 319 is realized by the GPU 237 and the CPU 234. A data storage section 320 stores the photographing control information output by the inference processing performed by the inference section 319. The data storage section 320 is realized by the HDD 235 or the like.

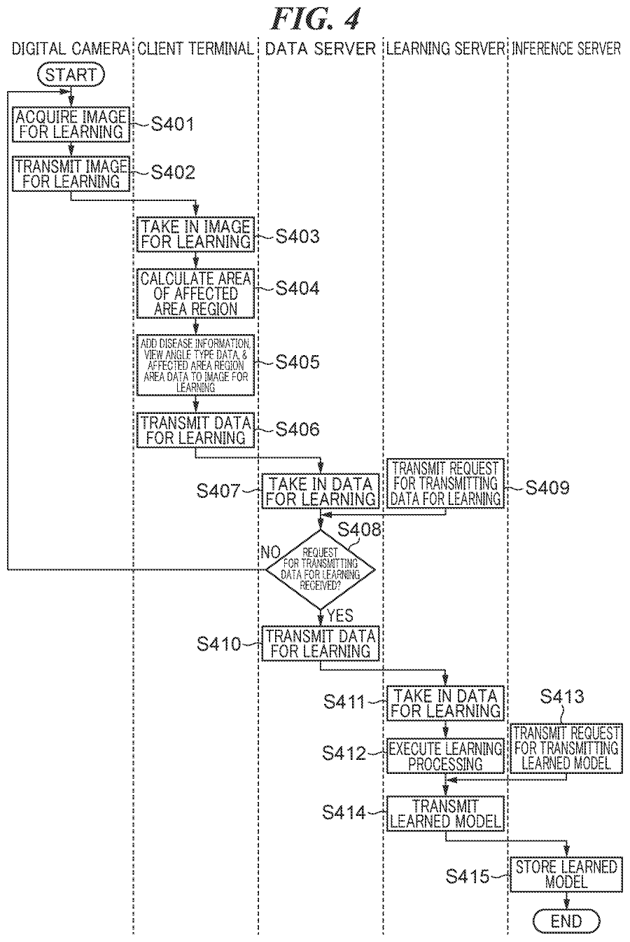

FIG. 4 is a flowchart of a learned model generation process performed in the photographing system 100 shown in FIG. 1. The learned model generation process in FIG. 4 is executed by the digital camera 101, the client terminal 102, the data server 105, the learning server 104, and the inference server 106. In FIG. 4, a process performed by the digital camera 101 is realized by the CPU 201 executing a program loaded in the RAM 203, a process performed by the client terminal 102 is realized by the CPU 211 executing a program loaded in the RAM 213, a process performed by the data server 105 is realized by the CPU 227 executing a program loaded in the RAM 229, a process performed by the learning server 104 is realized by the CPU 219 executing a program loaded in the RAM 221, and a process performed by the inference server 106 is realized by the CPU 234 executing a program loaded in the RAM 236.

Referring to FIG. 4, first, in a step S401, the CPU 201 of the digital camera 101 acquires an image for learning. More specifically, the CPU 201 causes the image sensor of the input section 206 to photograph an affected area to thereby generate an affected area image, and controls the data acquisition section 302 to acquire the affected area image as the image for learning. In the step S401, the CPU 201 acquires e.g. an overhead image captured from the whole affected area and an enlarged image captured from part of the affected area. Then, in a step S402, the CPU 201 controls the data transmission and reception section 303 to transmit the images for learning to the client terminal 102.

Then, in a step S403, the CPU 211 of the client terminal 102 takes in the images for learning, which are received from the digital camera 101. Then, in a step S404, the CPU 211 calculates an area of the affected area region in the image for learning. In the step S404, for example, the CPU 211 extracts the affected area region from the image for learning. Further, the CPU 211 calculates the area of the affected area region as a product of the number of pixels in the affected area region, and an area of each pixel, which is determined from a relationship between the angle of view of the digital camera 101 and an object distance.

Then, in a step S405, the CPU 211 adds disease information, view angle type data, and affected area region area data to the image for learning as metadata. The disease information is data acquired e.g. from an electronic medical record, such as a disease name and a patient ID for identifying a patient. The view angle type data is information on the angle of view of the affected area image which is the image for learning, more specifically, data indicating whether the image for learning is an overhead image or an enlarged image. For example, the CPU 211 performs image analysis on the image for learning using color information and the like, and in a case where there is a background area in the image for learning, the view angle type data is determined as the overhead image, and in a case where there is no background area in the image for learning, the view angle type data is determined as the enlarged image. Note that the configuration may be such that the user is prompted to input whether the image for learning is an overhead image or an enlarged image. The affected area region area data is data indicating an area of the affected area region, which is calculated in the step S404. In the following description, the image for learning to which the disease information, the view angle type data, and the affected area region area data are added as the metadata is defined as the data for learning. Then, in a step S406, the CPU 211 transmits the data for learning to the data server 105.

Then, in a step S407, the CPU 227 of the data server 105 takes in the data for learning, which is received from the client terminal 102. Further, the CPU 227 stores the data for learning in the HDD 228 or the like. Then, in a step S408, the CPU 227 determines whether or not a request for transmitting the data for learning has been received from the learning server 104. If it is determined by the CPU 227 that no request for transmitting the data for learning has been received from the learning server 104, the process returns to the step S401. Thus, in the present embodiment, the steps S401 to S408 are repeatedly executed to store a plurality of data items for learning in the HDD 228 until a request for transmitting the data for learning is received from the learning server 104.

In a step S409, the learning server 104 transmits a request for transmitting the data for learning to the data server 105, and if it is determined by the CPU 227 of the data server 105 in the step S408 that the request for transmitting the data for learning has been received from the learning server 104, the process proceeds to a step S410. In the step S410, the CPU 227 transmits all of the data items for learning, which are stored in the HDD 228, to the learning server 104.

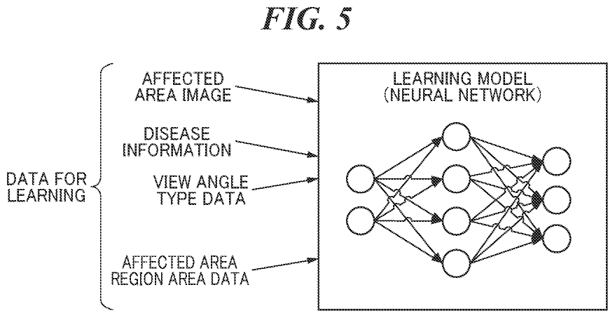

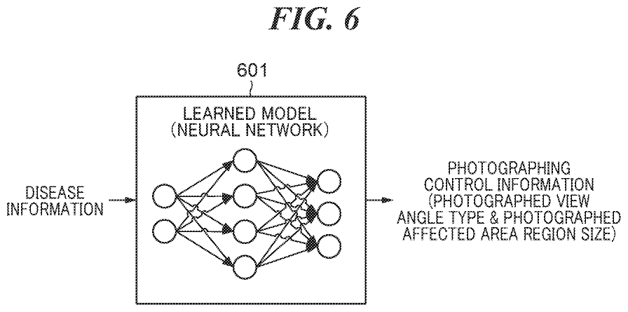

Then, in a step S411, the CPU 219 of the learning server 104 takes in the data for learning, which is received from the data server 105. Then, in a step S412, as illustrated in FIG. 5, the CPU 219 performs learning processing of a learning model using the data for learning, including the disease information, the affected area images, the view angle type data, and the affected area region area data. As a result, a learned model 601 shown in FIG. 6 is obtained. Note that the description of the present embodiment is given assuming that the learned model 601 is a neural network. However, the learned model 601 is not limited to the neural network. The learned model 601 is an inference model that infers the photographing control information using the disease information input thereto. The photographing control information includes a photographing view angle type and a photographed affected area region size. The photographing view angle type is information on the angle of view of the affected area image, and more specifically, information indicating whether the affected area image is an overhead image or an enlarged image. The photographed affected area region size is e.g. information indicating a ratio of the affected area region in the affected area image. Here, the photographed affected area region size will be described using an image obtained by photographing an affected area of a patient suffering from hives, as an example.

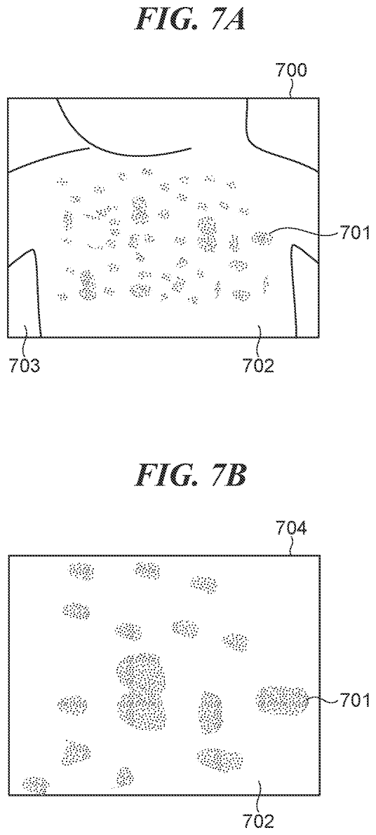

FIGS. 7A and 7B are views each showing an example of the affected area image obtained by photographing the affected area of the patient suffering from hives. An affected area image 700 shown in FIG. 7A is an overhead image captured from the whole affected area. An affected area image 704 shown in FIG. 7B is an enlarged image captured from part of the affected area. Reference numeral 701 denotes a rash area. Reference numeral 702 denotes a healthy area. Reference numeral 703 denotes a background area other than the rash area and the healthy area. Here, let it be assumed that the total area of all rash areas in the affected area image is represented by X, the total area of the healthy areas in the affected area image is represented by Y, and the total area of the background areas in the affected area image is represented by Z. Then, a photographed affected area region size W [%] is calculated by the following equation (1):

$$W = \frac{X}{X + Y + Z} \times 100 \qquad ( 1 )$$

For example, assuming, as for the affected area image 700, that X=240 [cm²], Y=750 [cm²], and Z=210 [cm²] hold, W=20 [%] is calculated from the equation (1). Further, assuming, as for the affected area image 704, that X=48 [cm²], Y=72 [cm²], and Z=0 [cm²] hold, W=40 [%] is calculated from the equation (1).

Referring again to FIG. 4, in a step S413, when the CPU 234 of the inference server 106 transmits a request for transmitting a learned model to the learning server 104, in a step S414, the CPU 219 of the learning server 104 transmits the learned model 601 to the inference server 106. In a step S415, the CPU 234 of the inference server 106 stores the learned model 601 received from the learning server 104 in the HDD 235. Then, the present process is terminated.

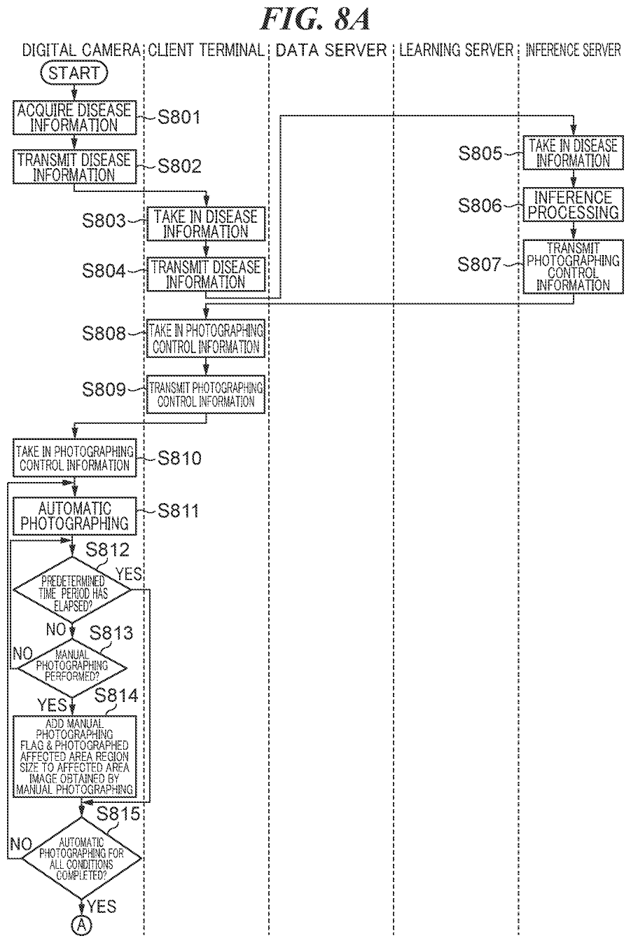

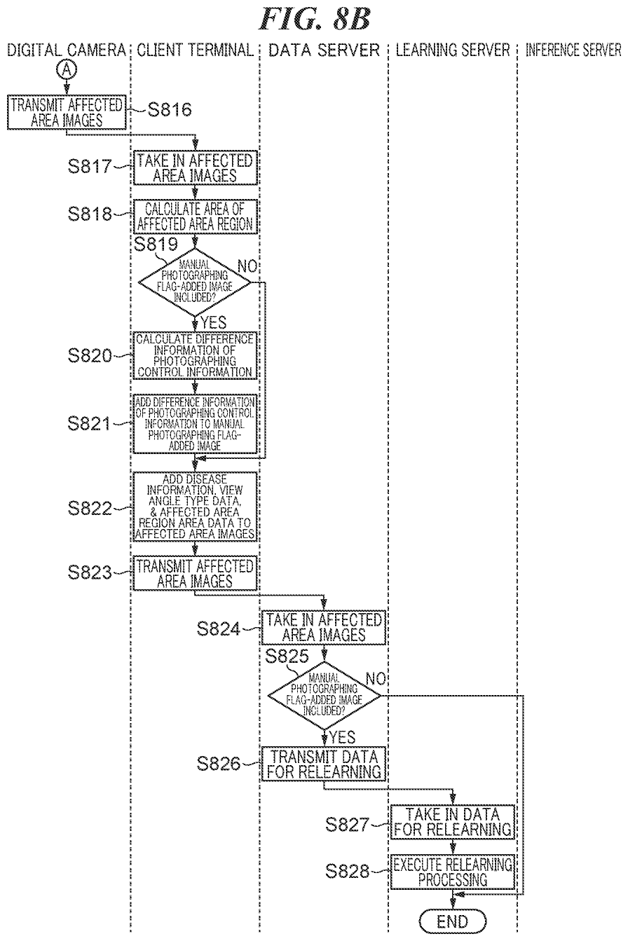

FIGS. 8A and 8B are a flowchart of a photographing control process performed in the photographing system 100 shown in FIG. 1. The photographing control process in FIGS. 8A and 8B is also executed by the digital camera 101, the client terminal 102, the data server 105, the learning server 104, and the inference server 106. In FIGS. 8A and 8B, a process performed by the digital camera 101 is realized by the CPU 201 executing a program loaded in the RAM 203, a process performed by the client terminal 102 is realized by the CPU 211 executing a program loaded in the RAM 213, a process performed by the data server 105 is realized by the CPU 227 executing a program loaded in the RAM 229, a process performed by the learning server 104 is realized by the CPU 219 executing a program loaded in the RAM 221, and a process performed by the inference server 106 is realized by the CPU 234 executing a program loaded in the RAM 236. Note that in FIGS. 8A and 8B, it is assumed that the above-described learned model generation process has been executed and the learned model 601 has already been stored in the HDD 235 of the inference server 106.

Referring to FIG. 8A, first in a step S801, the CPU 201 of the digital camera 101 acquires the disease information. For example, the CPU 201 reads a patient ID by reading a barcode and acquires, as the disease information, the patient ID and a disease name associated with the patient ID. Alternatively, the CPU 201 acquires, as the disease information, information input to the input section 206 by the user. Then, in a step S802, the CPU 201 transmits the disease information acquired in the step S801 to the client terminal 102.

Then, in a step S803, the CPU 211 of the client termina 1102 takes in the disease information received from the digital camera 101. Then, in a step S804, the CPU 211 transmits this disease information to the inference server 106.

Then, in a step S805, the CPU 234 of the inference server 106 takes in the disease information received from the client terminal 102. Then, in a step S806, the CPU 234 inputs the disease information to the learned model 601 stored in the HDD 235 and performs inference processing. By the inference processing, the photographing control information including the photographing view angle type and the photographed affected area region size is output. For example, in a case where “burn” is input to the leaned model 601 as the disease information, the photographing control information based on one condition is output. For example, the photographing control information including “overhead” as the photographing view angle type and “60%” as the photographed affected area region size is output. On the other hand, in a case where “hives” is input to the leaned model 601 as the disease information, respective items of the photographing control information based on two conditions are output. For example, an item of the photographing control information based on a first condition, including “overhead” as the photographing view angle type and “20%” as the photographed affected area region size for this photographing view angle type, is output. Further, an item of the photographing control information based on a second condition, including “enlarged” as the photographing view angle type and “40%” as the photographed affected area region size for this photographing view angle type, is output. Then, in a step S807, the CPU 234 transmits the photographing control information output in the step S806 to the client terminal 102.

Then, in a step S808, the CPU 211 of the client terminal 102 takes in the photographing control information received from the inference server 106. Then, in a step S809, the CPU 211 transmits the photographing control information to the digital camera 101.

Then, in a step S810, the CPU 201 of the digital camera 101 takes in the photographing control information received from the client terminal 102. Then, in a step S811, the CPU 201 performs automatic photographing based on the photographing control information. More specifically, the CPU 201 performs automatic photographing at the angle of view indicated by the photographing view angle type included in the photographing control information such that a ratio of the affected area region in the affected area image becomes the ratio indicated by the photographed affected area region size. Then, in a step S812, the CPU 201 determines whether or not a predetermined time period has elapsed after the automatic photographing in the step S811 has been performed. If it is determined by the CPU 201 that the predetermined time period has elapsed after the automatic photographing in the step S811 has been performed, the process proceeds to a step S815, described hereinafter. On the other hand, if it is determined by the CPU 201 that the predetermined time period has not elapsed after the automatic photographing in the step S811 has been performed, the process proceeds to a step S813.

In the step S813, the CPU 201 determines whether or not manual photographing has been performed. In the present embodiment, in a case where the attending doctor as the user determines that the affected area image obtained by the automatic photographing in the step S811 is unsuitable for diagnosis, the doctor rephotographs the affected area by manual photographing. The manual photographing refers to photographing performed such that the attending doctor adjusts the photographing control information taken in in the step S810 to other photographing control information, and performs photographing using the other photographing control information. If it is determined by the CPU 201 that the manual photographing has not been performed, the process returns to the step S812. On the other hand, if it is determined by the CPU 201 that the manual photographing has been performed, the process proceeds to a step S814.

In the step S814, the CPU 201 adds, as metadata, a manual photographing flag and a photographed affected area region size to an affected area image generated by the manual photographing. The manual photographing flag indicates that the affected area image has been generated by manual photographing. The photographed affected area region size is information indicating a ratio of the affected area region in the affected area image generated by manual photographing, and is calculated e.g. by the CPU 201. Then, in a step S815, the CPU 201 determines whether or not the automatic photographing is completed for all of photographing conditions. The photographing conditions corresponds to the condition(s) of the photographing control information taken in in the step S810. For example, in a case where the photographing control information based on a plurality of conditions is output by the inference processing performed by the inference server 106 as in the above-described case of hives, in the step S815, the CPU 201 determines whether or not the automatic photographing is completed with respect to all of these conditions. If it is determined in the step S815 that the automatic photographing is not completed with respect to all of the conditions, the process returns to the step S811, whereas if it is determined in the step S815 that the automatic photographing is completed with respect to all of the conditions, the process proceeds to a step S816 in FIG. 8B, wherein the CPU 201 transmits the affected area images to the client terminal 102. The affected area images transmitted in this step are affected area images obtained by excluding an affected area image determined to be unsuitable for diagnosis, from the affected area images generated by the automatic photographing. Further, in a case where manual photographing has been performed, these affected area images include affected area image(s) generated by the manual photographing.

Then, in a step S817, the CPU 211 of the client terminal 102 takes in the affected area images received from the digital camera 101. Then, in a step S818, similar to the step S404, the CPU 211 calculates the area of the affected area region. Then, in a step S819, the CPU 211 determines whether or not an affected area image to which the manual photographing flag has been added (hereinafter referred to as the “manual photographing flag-added image”) is included in the affected area images taken in in the step S817. If it is determined by the CPU 211 that no manual photographing flag-added image is included in the affected area images taken in in the step S817, the process proceeds to a step S822, described hereinafter. On the other hand, if it is determined by the CPU 211 that a manual photographing flag-added image is included in the affected area images taken in in the step S817, the process proceeds to a step S820.

In the step S820, the CPU 211 controls the arithmetic operation section 306 to calculate difference information of the photographing control information with respect to the manual photographing flag-added image. The difference information of the photographing control information refers to difference information between the photographing control information output by the inference processing performed by the inference server 106 and the other photographing control information used in the manual photographing. In the step S820, more specifically, the CPU 211 controls the arithmetic operation section 306 to calculate a difference between the photographed affected area region size output by the inference processing performed by the inference server 106 and the photographed affected area region size added to the manual photographing flag-added image as the metadata. Here, calculation of the difference information of the photographing control information will be described using an image obtained by photographing an affected area of a patient suffering from hives, as an example.

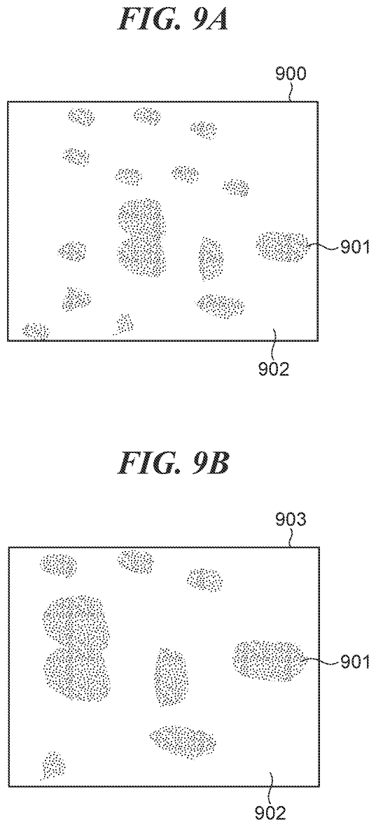

FIGS. 9A and 9B are views each showing an example of an enlarged image captured from part of the affected area of the patient suffering from hives. An affected area image 900 shown in FIG. 9A is an affected area image generated by the automatic photographing based on the photographed affected area region size output by the inference processing performed by the inference server 106. Let it be assumed that the photographed affected area region size in the affected area image 900 is represented by W AUTO rd. An affected area image 903 shown in FIG. 9B is an affected area image obtained by rephotographing the affected area by manual photographing. Let it be assumed that the photographed affected area region size in the affected area image 903 is represented by W_{MANUAL} [%]. A difference W_{DIFF} [%] in the photographed affected area region size is calculated by using the following equation (2):

W_{DIFF}=W_{MANUAL}−W_{AUTO} (2)

For example, assuming that W_{AUTO}=40[%] and W_{MANUAL}=50[%] hold, W_{DIFF}=10[%] is calculated by the equation (2).

Referring again to FIG. 8B, in a step S821, the CPU 211 adds, as metadata, the difference information of the photographing control information to the manual photographing flag-added image. Then, in the step S822, the CPU 211 adds, as metadata, the disease information, the view angle type data, and the affected area region area data to all of the affected area images. Then, in a step 823, the CPU 211 transmits all of the affected area images to the data server 105.

Then, in a step S824, the CPU 227 of the data server 105 takes in the affected area images received from the client terminal 102. Then, in a step S825, the CPU 227 determines whether or not a manual photographing flag-added image is included in the taken-in affected area images. If it is determined by the CPU 227 that no manual photographing flag-added image is included in the taken-in affected area images, the present process is terminated. On the other hand, if it is determined by the CPU 227 that a manual photographing flag-added image is included in the taken-in affected area images, the process proceeds to a step S826. In the step S826, the CPU 227 transmits the manual photographing flag-added image to the learning server 104 as data for relearning.

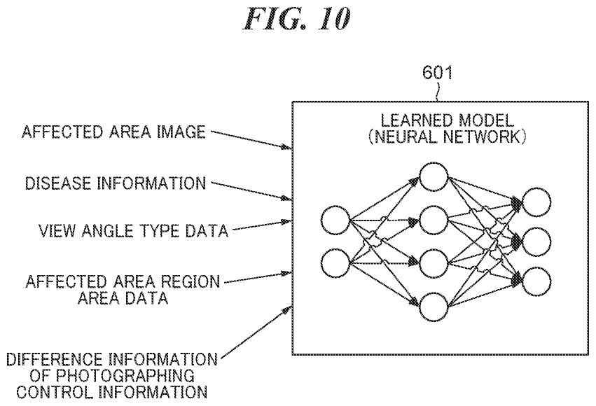

Then, in a step S827, the CPU 219 of the learning server 104 takes in the data for relearning, which is received from the data server 105. Then, in a step S828, as shown in FIG. 10, the CPU 219 performs relearning of the learned model 601 using the disease information, the affected area images, the view angle type data, the affected area region area data, and the difference information of the photographing control information, as the data for relearning. After that, the present process is terminated. The learned model 601 obtained by the relearning is transmitted from the learning server 104 to the inference server 106, and the inference server 106 stores the received learned model 601 in the HDD 235.

According to the above-described embodiment, in a case where an acquired affected area image is an image generated by manual photographing for performing photographing using the other photographing control information adjusted from the photographing control information, relearning of the learned model 601 is performed based on the information on the acquired affected area image. Through relearning of the learned model 601, in the next and subsequent photographing operations for the same disease, the photographing control information which makes it possible to obtain an affected area image suitable for diagnosis is transmitted to the digital camera 101, and the attending doctor is prevented from being required to manually rephotograph the affected area every time when photographing is performed for the same disease. This enables the attending doctor to perform efficient medical examination.

Further, in the above-described embodiment, the disease information includes a disease name. This makes it possible to transmit to the digital camera 101 the photographing control information that enables an affected area image that facilitates diagnosis to be obtained according to the disease name.

Furthermore, in the above-described embodiment, the disease information includes a patient ID. This makes it possible to transmit to the digital camera 101 the photographing control information that enables an affected area image that facilitates diagnosis to be obtained according to the patient ID.

In the above-described embodiment, to an image generated by manual photographing, the manual photographing flag indicating that the image has been generated by manual photographing is added as the metadata. With this, it is possible to easily identify the image generated by manual photographing, and it is possible to easily determine whether or not to perform relearning of the learned model 601.

The present invention has been described heretofore based on the embodiments thereof. However, the present invention is not limited to the above-described embodiments, but it can be practiced in a variety of forms, without departing from the spirit and scope thereof.

For example, although in the above-described embodiment, the photographing view angle type is classified into the two types, i.e. the overhead image and the enlarged image, this is not limitative, but the photographing view angle type may be classified into three types or more.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2022-117336 filed Jul. 22, 2022, which is hereby incorporated by reference herein in its entirety.

What is claimed is:

1. A photographing system that supports photographing of an affected area using an image capturing apparatus, comprising:
at least one processor; and
a memory coupled to the at least one processor, the memory having instructions that, when executed by the processor, perform the operations as:
a first acquisition unit configured to acquire photographing control information to be used by the image capturing apparatus to photograph the affected area, by inputting disease information transmitted from the image capturing apparatus to a learned model;
a transmission unit configured to transmit the photographing control information to the image capturing apparatus;
a second acquisition unit configured to acquire an affected area image generated by the image capturing apparatus photographing the affected area; and
a relearning unit configured to perform, in a case where the acquired affected area image is an image generated by manual photographing for performing photographing using other photographing control information adjusted from the photographing control information, relearning of the learned model based on information on the acquired affected area image.

2. The photographing system according to claim 1, wherein the disease information is a disease name.

3. The photographing system according to claim 1, wherein the disease information is a patient ID for identifying a patient.

4. The photographing system according to claim 1, wherein the learned model is generated by learning using disease information, an affected area image, information on an angle of view of the affected area image, and information indicating an area of an affected area region in the affected area image.

5. The photographing system according to claim 1, wherein the photographing control information includes information on an angle of view of an affected area image and information indicating a ratio of an affected area region in the affected area image.

6. The photographing system according to claim 1, wherein the acquired information on the affected area image includes at least difference information between the photographing control information and the other photographing control information.

7. The photographing system according to claim 1, wherein, to an image generated by the manual photographing, a flag indicating that the image is generated by the manual photographing is added as metadata.

8. A photographing control method for supporting photographing of an affected area using an image capturing apparatus, comprising:
acquiring photographing control information to be used by the image capturing apparatus to photograph the affected area, by inputting disease information transmitted from the image capturing apparatus to a learned model;
transmitting the photographing control information to the image capturing apparatus;
acquiring an affected area image generated by the image capturing apparatus photographing the affected area; and
performing, in a case where the acquired affected area image is an image generated by manual photographing for performing photographing using other photographing control information adjusted from the photographing control information, relearning of the learned model based on information on the acquired affected area image.

9. A non-transitory computer-readable storage medium storing a program for causing a computer to execute a photographing control method for supporting photographing of an affected area using an image capturing apparatus,
wherein the photographing control method comprises:
acquiring photographing control information to be used by the image capturing apparatus to photograph the affected area, by inputting disease information transmitted from the image capturing apparatus to a learned model;
transmitting the photographing control information to the image capturing apparatus;
acquiring an affected area image generated by the image capturing apparatus photographing the affected area; and
performing, in a case where the acquired affected area image is an image generated by manual photographing for performing photographing using other photographing control information adjusted from the photographing control information, relearning of the learned model based on information on the acquired affected area image.
